(19) United States
(12) Patent Application Publication
(10) Pub. No.: US 2002/0034553 A1
(43) Pub. Date: Mar. 21, 2002

(54) Skin healing, conditioning and enhancing lotion

(76) Inventor: Janice Zayas, Torrance, CA (US)

Correspondence Address: Roger A. Marrs, Suite 1120, 15233 Ventura Blvd., Sherman Oaks, CA, 91403, US

(21) Appl. No.: 09/955,908

(22) Filed: Sep. 19, 2001

Related U.S. Application Data
(60) Provisional application No. 60/233,715, filed on Sep. 19, 2000.

(51) Int. Cl. A61K 35/78; A61K 33/40

(52) U.S. Cl. 424/614; 424/616; 424/744; 424/762

(57) ABSTRACT

A combination of ingredients and a method of combining the ingredients wherein the resultant product improves the healing of skin tissues, speeds healing time and enhances the skin's revitalization. The basic ingredients include a predetermined combination of food grade hydrogen peroxide combined with certified organic aloe vera gel and other ingredients chosen from a variety of substances, such as ultra-purified distilled water and Irish moss.

### BACKGROUND OF THE INVENTION

1. Field of the Invention

The present invention relates to the field of medicinal skin products, and more particularly to a novel and unique blend of ingredients in the form of a stable, aqueous gel incorporating hydrogen peroxide that is particularly useful as an antiseptic in healing bedsores, easing pain and which enhances the skin's texture, condition and well being.

2. Brief Description of the Prior Art

In the past, there have been a variety of ointments, creams and lotions which were employed to improve the condition of a person's skin. In some instances, conventional ointments or creams are used for not only cleansing and revitalizing but for medicinal purposes in improving healing time of wounds and/or reducing or eliminating scarring. Problems and difficulties have been encountered when using such conventional products, which stem from the fact that such skin products do not include oxygen-carrying ingredients in their combination and further conventional products lack some of the ingredients which are necessary for healing, conditioning and revitalizing skin tissue.

For example, bedsores can evolve into decubitus ulcers and these ulcers may cause a great deal of pain which eventually may evolve into a life-threatening situation. Generally, these ulcers are caused by a lack of sufficient oxygen due to a closed environment, poor ambient circulation or tight dressings. Decubitus ulcers or pressure sores, as such are commonly referred to, are localized areas of infractured soft tissues reduced by pressure. Pressure is exerted on the skin and subcutaneous tissues by the object on which a patient must rest, such as a mattress, a chair seat, a cast or the like. There is compression of the small nutrient vessels of the skin and underlying tissues which results in tissue anoxia or ischemia. The cutaneous tissues become broken or destroyed which leads to progressive destruction of underlying soft tissue. Once the skin breaks, an ulcer may form which can be painful and very slow to heal.

Other skin tissue problems are encountered, such as cuts, abrasions, burns and in treating gingivitis pyorrhea.

Attempts have been made to provide skin tissue healing formulas or ointments which are disclosed in U.S. Pat. No. 4,725,438. However, such a disclosure only relates to an aloe vera ointment useful in treating skin irritations and promoting healing of wounds without providing oxygen to the tissue area. Also, U.S. Pat. No. 5,260,021 discloses a hydrogen peroxide-containing gel ointment as a vehicle for carrying oxygen; however, such a disclosure is for non skin treatment and is employed for use only as a disinfectant for contact lenses or the like. In U.S. Pat. No. 4,696,757, a hydrogen peroxide gel is disclosed which is useful in treating surface cuts and for bleaching hair but does not include skin revitalizing ingredients such as aloe vera or the like.

Therefore, a long-standing need has existed to provide a unique blend of ingredients which will promote healing of skin tissue and which will supply oxygen to an affected area for decreasing pain, preventing the spread of infection and for healing of the tissue. Such a blend of ingredients should include a combination which is sterile and which may include a predetermined combination of food grade hydrogen peroxide, ultra-purified water, certified organic aloe vera gel and Irish moss.

### SUMMARY OF THE INVENTION

Accordingly, the above problems and difficulties are avoided by the present invention which provides for a combination of ingredients and a method of combining the ingredients so that the resultant product improves the healing of skin tissues as well as healing time and which enhances the skin's revitalization. The basic ingredients include a predetermined combination of food grade hydrogen peroxide combined with certified organic aloe vera gel and with other ingredients chosen from a variety of substances, such as ultra-purified distilled water and Irish moss. If desired, natural herbs, vitamins or the like may be included in the combination.

Therefore, it is among the primary object of the present invention to provide a unique blend or combination of available ingredients that will heal skin tissue, reduce healing time and promote revitalization of skin tissue.

Another object of the present invention is to provide a novel healing and skin tissue enhancement product which diminishes scarring, promotes healing, decreases pain and prevents the spread of infection.

Still a further object of the present invention is to provide a novel formulation which supplies oxygen to an affected skin tissue area and further promotes healing of the tissue and a decrease in pain while preventing the spread of infection.

A further object resides in providing a healing formula which is not only effective for the treatment of bedsores and the relief of pain but which is effective in other areas, such as the healing of cuts, abrasions, burns and in the treatment of gingivitis pyorrhea.

### DESCRIPTION OF THE PREFERRED EMBODIMENT

In accordance with the present invention, the inventive formula or combination of ingredients is for aiding in healing and promotion of recovery from such skin conditions as decubitus, ulcers, burns and possible gangrene. The inventive formula works by supplying oxygen to the affected area to promote healing, decrease pain, and prevent the spread of infection. The inventive formula may also be effective in treating gingivitis pyorrhea. Through the process of oxidizing the infected or irritated area, the level of pain is also reduced.

Specifically, the inventive healing formula is produced using a predetermined combination of 35% food grade hydrogen peroxide, distilled water, certified organic aloe vera gel and Irish moss herb. In the case of usage for ulcers, it is noted that the ulcers tend to resist ordinary forms of treatment but application of hydrogen or zinc peroxide liberates oxygen over a long period of time to convert the anaerobic portions of a wound into an aerobic area. Healing occurs rapidly owing to the inability of the aerobic streptococci to live in an unfavorable environment. The inventive healing formula utilizes a combination of oxygen liberating agents with topical agents to promote the healing of serious and sometimes life-threatening conditions.

The specific ingredients include ultra-purified water or distilled water in the amount of 11 parts mixed with one part hydrogen peroxide and mixed with 99.6% pure aloe vera in the amount of 12 parts. Within the 12 parts is also mixed Irish moss, scorbic acid, ascorbic acid, and citric acid. The aloe vera primarily forms a gel holding the ingredients together in an ointment or lotion which may be applied directly to a cleansed infected or irritated skin tissue area. Such application may be by direct application, spraying, brushing, saturated dressing or the like.

Initially, the user's skin is first tested by applying the inventive healing formula in a cream or lotion form to a selected skin area. The user waits to see if any adverse effects, such as burning, allergy reaction or the like, may occur. If the test proves positive for the user to continue, then the skin is thoroughly cleansed with soap and water or cleanser followed by applying a toner to the area requiring healing. The toner may take the form of a witchhazel solution or even a buffered saline solution. The inventive healing formula may be directly applied to the cleansed area and preferably, the use of the healing formula should be applied twice a day. The healing formula should be refrigerated after opening and should be thoroughly shaken before using.

For use in connection with bedsores, decubitus ulcers, the health care person should use sterile gloves and should flush out the decubitus with a syringe filled with sterile water. Following this procedure, the decubitus is removed by wiping away with a soft sterile gauze and sufficient gauze is cut to fill the decubitus. Next, the health care provider puts 2-3 days formula in a small bowl or jar with a lid that is sterile. Next, the cut-off gauze is soaked in a bowl with a formula and excess liquid is squeezed out. Carefully, the gauze is stuffed into the decubitus completely to the top of the wound being careful not to pack tightly. A dressing is employed to cover the decubitus by using a sterile patch and with medicinal paper tape to seal the patch to the skin on all sides. This procedure should be accomplished morning and evening.

For cuts, abrasions and burns, the injury is initially cleaned with the inventive healing formula which is applied liberally every 2-3 hours. In more severe injuries, a patch is soaked with the inventive healing formula and it is placed over the wound followed by sealing with medicinal paper tape. The procedure should be performed twice daily.

As a use for mouthwash, the inventive healing formula is very good for pyorrhea conditions. With a trickle bottle, the teeth and gums are thoroughly brushed or massaged with the healing formula. Next, the mouth is rinsed with pure water making sure not to swallow.

While particular embodiments of the present invention have been shown and described, it will be obvious to those skilled in the art that changes and modifications may be made without departing from this invention in its broader aspects and, therefore, the aim in the appended claims is to cover all such changes and modifications as fall within the true spirit and scope of this invention.

What is claimed is:

1. A healing formula for direct application to the skin of a user comprising:
a predetermined combination of ingredients including:
at least 35% food grade hydrogen peroxide;
distilled water added to said food grade peroxide;
certified organic aloe vera gel; and
Irish moss herb mixed with said water, said food grade peroxide and said aloe vera gel.

2. The formula defined in claim 1 wherein:
said food grade peroxide is chosen from:
a. Hydrogen peroxide;
b. Zinc peroxide.

3. The formula defined in claim 2 wherein:
said food grade peroxide is an oxygen liberating agent effective to convert the anaerobic portions of a wound into an aerobic area.

4. The formula defined in claim 3 wherein:
said combination of ingredients creates a healing of the wound owing to the inability of anaerobic streptococci to live in an unfavorable environment.

5. The formula defined in claim 3 wherein:
said combination of ingredients establishes an unfavorable environment in which anaerobic streptococci cannot live.

6. A healing formula for oxidizing an infected or irritated skin area comprising:
a predetermined combination of ingredients;
said ingredients consisting of:
a. food grade peroxide, hydrogen or zinc.
b. distilled water;
c. certified organic aloe vera gel; and
d. Irish moss herb.

7. The healing formula defined in claim 6 wherein:
said distilled water is ultra-purified in the amount of 11 parts mixed with one part of said food grade peroxide and further mixed with 99.6% pure aloe vera in an amount of 12 parts and said amount of 12 parts containing a mixture of Irish moss, ascorbic acid and citrus acid.

8. The healing formula defined in claim 7 wherein:
said aloe vera gel is a binder holding said combination of ingredients together.

9. A healing formula for oxidizing an infected or irritated skin area comprising:
an ultra-purified water constituting an amount of 11 parts to a total formulation;
hydrogen peroxide mixed with said 11 parts in an amount of 1 part to said total formulation;
pure aloe vera mixed with said 11 parts of said ultra-purified water and said 1 part of hydrogen peroxide in an amount equal to 99.6% of said total formulation; and
Irish moss mixed with a trace quantity of ascorbic acid included in said total formulation.

10. The formula defined in claim 9 wherein:
said pure aloe vera constitutes a gel as a binder to provide said total formulation in a lotion for direction application to the skin area of the user.
